(19) United States
(12) Patent Application Publication
(10) Pub. No.: US 2006/0009640 A1
(43) Pub. Date: Jan. 12, 2006

(54) Preparation of (-)-galantamine hydrobromide

(76) Inventors: Vijaya Bhaskar Bolugoddu, Hyderabad (IN); Sanjay Shukla, Agra (IN); Mukunda Reddy Jambula, Mahbubnagar (IN); Rajeshwar Reddy Sagyam, Mahbubnagar (IN); Ramchandra Reddy Pingili, Hyderabad (IN); Ananda Babu Thirunavakarasu, Chennai (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/177,897

(22) Filed: Jul. 8, 2005

Related U.S. Application Data
(60) Provisional application No. 60/586,430, filed on Jul. 8, 2004.

(51) Int. Cl. A61K 31/55; C07D 491/02

(52) U.S. Cl. 540/581

(57) ABSTRACT

A process for preparing (−)-galantamine hydrobromide.

### CROSS-REFERENCE TO RELATED APPLICATION

This application claims priority from copending U.S. Provisional Application No. 60/586,430 filed Jul. 8, 2004, the entire content of which is hereby incorporated by reference.

### INTRODUCTION TO THE INVENTION

The present invention relates to a process for the preparation of galantamine hydrobromide, which is chemically known as (4aS,6R,8aS)-4a,5,9,10,11,12-hexahydro-3-methoxy-11-methyl-6H-benzofuro-[3a,3,2-ef][2]-benzazepin-6-ol hydrobromide, and which can be represented by the Formula (I).

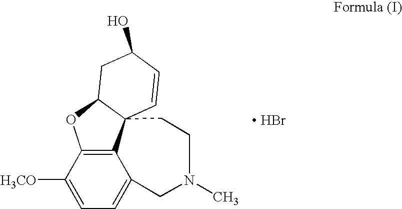

Galantamine, also known in the literature as galanthamine, is a tertiary alkaloid, which, owing to its pharmacological properties, belongs to the group of reversibly acting acetylcholinesterase inhibitors. Its effects are similar to those of phytostigmine and neostigmine. Galantamine is used in poliomyelitis and different diseases of the nervous system, but mainly in the treatment of narrow-angle glaucoma and as antidote after curare applications. Galantamine is therefore useful pharmacologically for paralysis symptoms resulting from polio mellitus and for different diseases of the nervous system. Galantamine hydrobromide is being sold in pharmaceutical products for treating Alzheimer's disease, using the trademark REMINYL.

Galantamine is usually isolated from plants belonging to Amaryllidaceae alkaloids, for example, from galanthus species, such as the snowdrop or Leucojum aestivum. These plants having galantamine in concentrations of up to 0.3% with only small amounts of companion alkaloids so that the extraction method described in DE-PS 11 93 061 can be used. This process of extraction is not feasible to practice at an industrial scale.

Apart from plant sources, a chemical process for the synthesis of galantamine and its analogues including its acid addition salts, has been disclosed in International Published Application No. WO 95/27715.

D. H. R. Barton et al., in Journal of the Chemical Society, pages 806-817, 1962 describes the process for making derivatives and precursor of galantamine and is prepared by oxidative cyclization of N-(3-hydroxy-4-methoxyphenyl)-N-methyl-4-hydroxy-phenylethylamine. Since the yields are very low, galantamine cannot be prepared as commercial batches using this process.

R. A. Holton et al., in Journal of the American Chemical Society, Vol. 110, pages 314-316, 1988 describes a process for the preparation of narwedine from isovanillin in an overall yield of 44%. The major drawbacks of the described process are usage of expensive and hazardous reagents such as palladium and also thallium trifluoroacetate in an equimolar amount, and the lengthy process of production.

U.S. Pat. No. 6,407,229 describes a process for the preparation of galantamine in which bromoformyl-narwedine is reduced with reducing agents like L-Selectride. The reduction leads diastereoselectively to N-dimethyl bromo galantamine, which can be converted into (±) galantamine by N-methylation according to Eschweiler-Clark and de-bromination.

U.S. Pat. No. 6,392,038 describes a process for the preparation of a substantially single enantiomer of (−)-galantamine, prepared from (−)-Narwedine having enantiomeric excess of at least 80%, and on further reducing giveing (−)-galantamine.

Several procedures have been developed for the resolution of galantamine. One procedure involves formation of a diastereomeric salt with di-para-toluoyl tartaric acid and separation of the mixture by recrystallisation (Heterocycles, 1976, 1111). However, preparation of therapeutically active (−)-galantamine renders this process costly.

According to Journal of Heterocyclic Chemistry (1995) 32: 195, another resolution procedure involves the formation of diastereomeric esters with (−)-camphanic chloride and separation of the mixture by recrystallisation. The resultant product is then converted into (−)-galantamine by reduction in a process, which destroys the chiral auxiliary group, so rendering this process impractical for economic production.

A need remains for a cost effective, safe, and industrially feasible route to synthesize highly enantiomerically pure galantamine hydrobromide and an invention made toward this goal is described below.

### SUMMARY OF THE INVENTION

A process for preparing galantamine hydrobromide involves preparing useful intermediate compounds having following Formula (IVC), Formula (IVD), Formula (IVE), Formula (V), Formula (VI) and Formula (VII) and their subsequent conversion to galantamine hydrobromide of Formula (I).

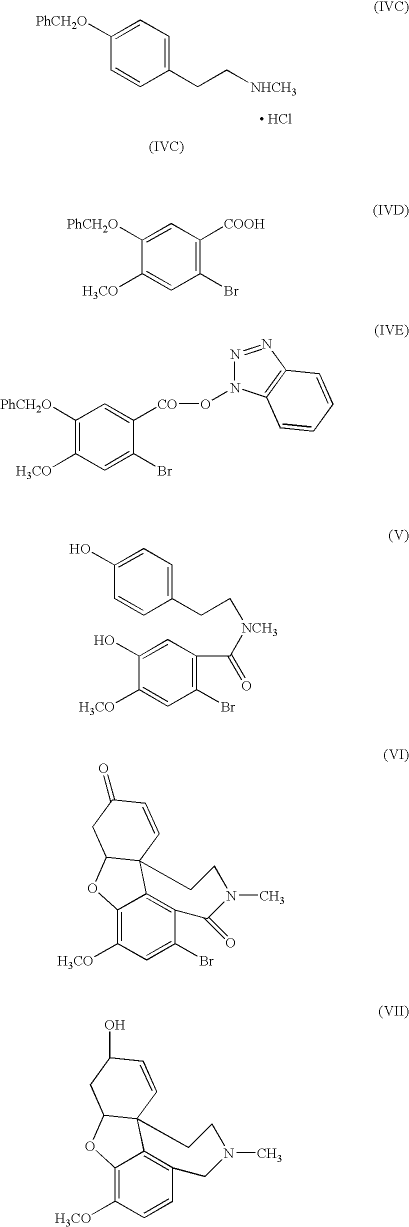

A process for the preparation of the compound of Formula (V) involves reacting a compound of Formula (IVD) with 1-hydroxy benzotriazole (HOBT) and dicyclohexyl carbodiimide (DCC) in the presence of dichloromethane, giving the HOBT complex compound of Formula (IVE). Further condensing the compound of Formula (IVE) with the compound of Formula (IVC) in the presence of alkaline hydroxides like sodium hydroxide and in alcoholic medium gives the compound of Formula (Va). The compound of Formula (Va) on further hydrolysis in the presence of acid or base and methyl isobutyl ketone (MIBK) gives the desired compound of Formula (V) as shown in the following Scheme 1.

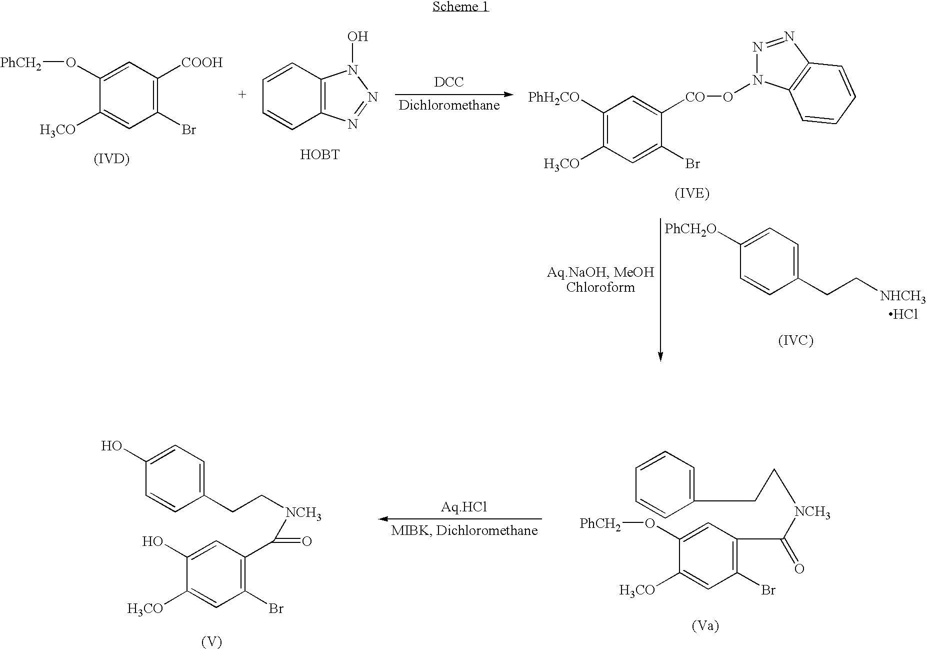

A starting compound of Formula (IVC) can be prepared by the process involving reacting the compound of Formula (IIC) with aqueous methylamine and isolating in ethyl acetate to give para-benzyloxy phenyl acetic acid N-methyl amide of Formula (IIIC). Further reactions include converting the compound of Formula (IIIC) into the compound of Formula (IVC) by reacting with sodium borohydride in the presence of acetic acid and dioxane, further reacting with a solution alcoholic HCl (HCl in isopropyl alcohol) in the presence of halogenated hydrocarbon solvents like chloroform, and finally isolating the desired compound as represented in the following Scheme 2.

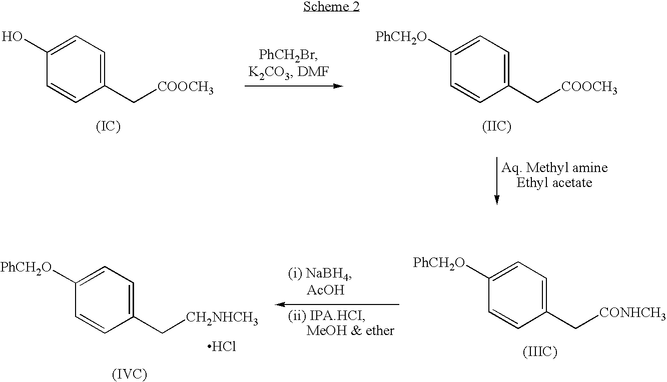

The starting compound of Formula (IIC) can be prepared from its corresponding hydroxy compound by reacting with benzyl halide in presence of potassium carbonate and dimethylformamide.

Another starting compound of Formula (IVD) of Scheme-1 can be prepared by reacting isovanilin (ID) with benzyl halide in the presence of potassium carbonate and dimethyl formamide, to obtain benzyl protected isovanilin (IID), which on further bromination with bromine in the presence of alcohols provides bromo compound (IIID), which can be purified by crystallization from ethyl acetate. The compound (IIID) is further oxidized with potassium permanganate in the presence of acetone to obtain compound (IVD) as represented in the following Scheme 3.

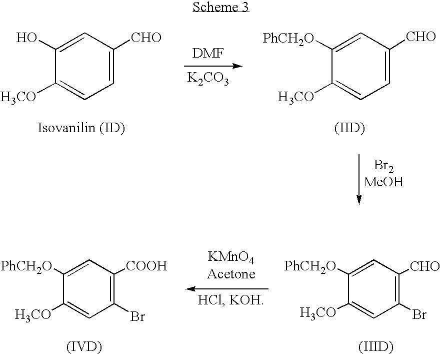

Further, a process for the preparation of galantamine hydrobromide also involves preparation of an intermediate compound of Formula (VII), by reacting the compound of Formula (VIA) with VITRIDE (VITRIDE is also called “Red-Al” and is chemically sodium dihydro-bis-(2-methoxyethoxy) aluminate or sodium aliminum bis (2-methoxyethoxy) hydride, sold in a ready-to-use form, as a 70 percent solution in toluene, by Rohm and Haas Company, Philadelphia, Pa. U.S.A.) and finally isolating the desired compound, as represented in the following Scheme 4.

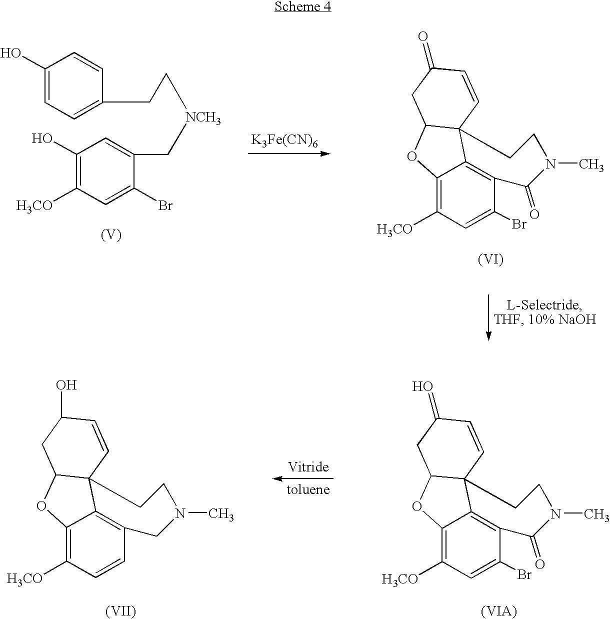

One more embodiment of the present invention is in the preparation of the compound of Formula (VI) by cyclization of compound of Formula (V) either by incremental or one-time addition of cyclizing reagent in the presence of bicarbonate, for improving product yield. Final purification of the compound of Formula (VI) by recrystallization provides the required compound in good yield.

An aspect of the invention is providing (−)-galantamine hydrobromide containing: no more than about 0.2 percent by weight of an impurity having formula (VIII), as measured by high performance liquid chromatography; no more than about 0.15 area-percent, or no more than about 0.05 area-percent, as measured by high performance liquid chromatography, of the impurities 1,2-dihydrogalantamine or 3,4-dihydrogalantamine (lycoramine) of formula (IX); no more than about 0.15 area-percent, or no more than about 0.09 area-percent, as measured by high performance liquid chromatography, of the impurity (4a,6a)-4a,5,9,10,11,12-hexahydro-3-methoxy-11-methyl-6H-benzofuran[3a,3,2-ef][2]benzazepine-6-ol (epi-galantamine) of formula (X); no more than about 0.15 area-percent, or no more than about 0.03 area-percent, as measured by high performance liquid chromatography, of the impurity which is the N-oxide of (4a,6a)-4a,5,9,10,11,12-hexahydro-3-methoxy-11-methyl-6H-benzofuran[3a,3,2-ef][2]benzazepine-6-ol and having formula (XI); or no more than about 0.15 area-percent, or no more than about 0.03 area-percent, as measured by high performance liquid chromatography, of the impurity (4 as,6R,8 as)-4a,5,9,10,11,12-hexahydro-3-methoxy-6H-benzofuro[3a,3,2-ef][2]benzazepin-6-ol (N-desmethyl galantamine) having formula (XII). In general it is desired that the total impurities will not exceed about 1 area-percent, or about 0.3 area-percent.

### BRIEF DESCRIPTION OF THE DRAWING

FIG. 1 is an X-ray powder diffractogram (XRPD) of galantamine hydrobromide, prepared according to Example 12.

### DETAILED DESCRIPTION OF THE INVENTION

Referring to the preceding Scheme I, the compound of Formula (V) can be prepared by reacting 5-benzyloxy-2-bromo-4-methoxybenzoic acid of Formula (IVD) with 1-hydroxybenzotriazole (HOBT) and dicyclohexylcarbodiimide (DCC), wherein the solvents can be a halogenated hydrocarbon such as dichloromethane, dichloroethane or chloroform, or any mixture thereof, or an ester solvent like ethyl acetate, at about 10 to 70° C. for 3 to 5 hours, followed by filtering out undissolved solids by conventional methods. The resultant compound obtained by the process can be purified by recrystallazation from ketonic solvents such as acetone, methyl isobutyl ketone, propanone, butanone, or any mixture thereof, or an alcohol such as methanol, ethanol, propanol, or any mixture thereof, by controlled heating and cooling cycles between 0 and 70° C. Solids are separated, such as by filtration, at 0-10° C. and then dried at 20-70° C. under reduced pressure. The obtained solids are further reacted with parabenzyloxy-N-methylphenethylamine hydrochloride of formula (IVC) in the presence of an alkali metal hydroxide such as sodium hydroxide or potassium hydroxide, in the presence of an organic solvent, wherein the solvent can be a halogenated hydrocarbon such as dichloromethane, dichloroethane, chloroform, or any mixture thereof, or an ester solvent such as ethyl acetate, and the obtained compound on further hydrolysis in the presence of an acid such as hydrochloric acid, hydrobromic acid, or a base such as sodium hydroxide or potassium hydroxide, in the presence of a ketonic solvent such as acetone, methyl isobutyl ketone, propanone, butanone, or any mixture thereof, or an alcohol such as methanol, ethanol, or propanol, or other solvents like water or acetonitrile, or any mixture of such solvents, gives the desired compound.

Para benzyloxy-N-methyl phenethyl amine HCl of formula (IVC) can be prepared by mixing para benzyloxy phenyl acetic acid methyl ester of Formula (IIC) with mono methyl amine at about 0-50° C. and stirring for about 3-7 hours. Filtering the solution to separate solids, followed by washing with water and then drying the solids for about 5-10 hours at about 45-55° C. under reduced pressure, and optionally recrystallization using solvents such as ethyl acetate, methyl acetate, or isopropyl acetate, preferably ethyl acetate, results in the compound of formula (IIIC).

The obtained compound of formula (IIIC) is dissolved in 1,4-dioxane at 5 to 40° C. and sodium borohydride is added and the mixture is stirred at 5 to 40° C. for about 20 to 30 minutes. A solution of acetic acid and 1,4-dioxane is added to the above solution at about 5 to 40° C., then the mixture is heated to 50 to 75° C. followed by stirring for about 1 to 10 hours. The organic solvent is then distilled off under reduced pressure and the compound (IVC) is isolated from water.

The resulting crude compound (IVC) can be purified by dissolving in a halogenated hydrocarbon solvent such as dichloromethane or chloroform, addition of a solution of hydrochloric acid in isopropyl alcohol at about 0-40° C., and stirring for about 3 to 15 hours. Distilling off the solvent under reduced pressure followed by addition of diethyl ether and methanol at about 0-10° C. results in the formation of solids that are filtered and washed with ether, and then dried under reduced pressure to produce the pure compound of formula IVC.

The compound of Formula (IVD) can be prepared by reacting isovanilin (ID) with benzyl bromide in an aprotic solvent such as dimethyl formamide or dimethyl sulfoxide and in the presence of a base such as sodium carbonate, potassium carbonate, sodium hydroxide, potassium hydroxide, potassium tertiary butoxide, sodium bicarbonate and a solvent such as dimethylformamide, dimethyl sulfoxide or an aprotic solvent, to give benzyl-protected isovanilin, which is then brominated with bromine or a source of bromine in the presence of an alcoholic solvent such as methanol, ethanol, propanol, tertiary butanol or a halogenated hydrocarbon such as dichloromethane, dichloroethane or chloroform, or any mixture thereof, or an ester solvent such as ethyl acetate, and the obtained bromo compound is oxidized with an oxidizing agent like potassium permanganate in a keto solvent such as acetone, propanone or methyl isobutyl ketone.

The compound of Formula (VIA) can be prepared by reacting a compound of formula (VI) (Bromo Narwedenone) with L-SELECTRIDE (a 1M solution of lithium tri-sec-butylborohydride in tetrahydrofuran, sold by Sigma-Aldrich of St. Louis, Mo. U.S.A.) in the presence of tetrahydrofuran (THF) at about −70 to 30° C., and stirring the resulting reaction mass for about 1 to 5 hours at the same temperature. Decomposing the reaction mixture is accomplished by adding water at about −10 to 30° C., followed by hydrogen peroxide, and then 10% sodium hydroxide solution added to the reaction mixture at about −10 to 30° C. The mixture is extracted with dichloromethane followed by washing the combined organic layers with sodium bicarbonate solution, and the solvent is distilled under reduced pressure, followed by addition of aqueous sodium hydroxide solution to the residue and stirring for about 2 to 6 hours. The resulting solids are filtered, washed and dried under reduced pressure.

The solid of Formula (VIA) is dissolved in an aromatic hydrocarbon solvent such as toluene or xylene at reflux temperature, and to this a solution of VITRIDE in an aromatic hydrocarbon solvent such as toluene or xylene is added at reflux temperature. The resulting reaction mass is refluxed for about 1 to 2 hours followed by cooling and 15% sodium potassium tartrate solution is added to the cooled reaction mass and maintained for about 1-3 hours at about 0-30° C. The solution is filtered through a perlite bed followed by washing with an aromatic hydrocarbon solvent such as toluene or xylene. The separated organic solution is washed with water followed by sodium chloride solution and distilling off the organic solvent under reduced pressure to produce solids of the compound of Formula (VII). The obtained compound having Formula (VII) is further purified in methyl tertiary butyl ether and dichloromethane.

Further converting the obtained compound of racemic galantamine of Formula (VII) to the compound galantamine hydrobromide of Formula (I) is performed by a sequence of reactions, comprising resolution of the compound of Formula (VII) by treating with (+)-di-para-toluoyl tartaric acid in methanol to give the tartrate salt of (−)-galantamine of Formula (VII) which is further converted to (−)-galantamine HBr, and converting the compound (−)-galantamine HBr to its free base by reacting with aqueous sodium carbonate solution in aromatic hydrocarbon solvents. Then, the obtained pure galantamine free base is reacted with 30 to 50% hydrobromic acid in ethanol to give the final compound (−)-galantamine hydrobromide of Formula (I)

(−)-Galantamine hydrobromide prepared according to this embodiment has a low level of impurities as determined by HPLC. For example, it contains about 0.15 area-percent, or 0.05 area-percent, or less, of lycoramine of formula (IX), 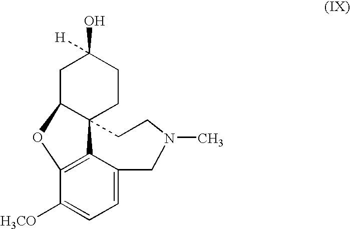
about 0.15 area-percent, or 0.09 area-percent, or less, of Epi-galantamine of formula (X), 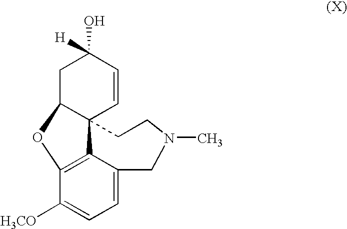
about 0.15 area-percent, or 0.03 area-percent, or less, of the N-oxide of formula (XI), 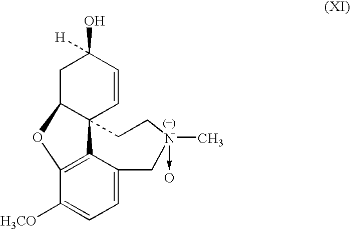
about 0.15 area-percent, or 0.03 area-percent, or less, of N-desmethyl galantamine of formula (XII), and 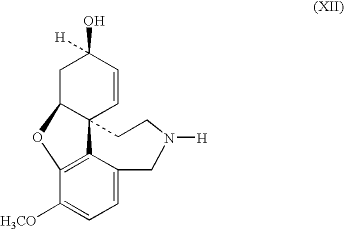
about 1 area-percent, or 0.3 area-percent, or less, of total impurities.

For use in pharmaceutical compositions, the galantamine hydrobromide will frequently be reduced to small particle sizes, such as by milling. In general, the mean particle size will be less than about 50 μm, or less than about 25 μm, or less than about 5 μm. Particle size distribution will frequently be such that the D₉₀ is less than about 25 μm, or about 8 μm, the D₅₀ will be less than about 15 μm, or less than about 3 μm, and the D₁₀ will be less than about 5 μm, or less than about 0.7 μm. The “D” value is a measure of the volume percentage of particles, as determined by a Malvern particle size method, having sizes less than the indicated size. For example, a D₉₀ of 25 μm means that 90 percent of the particles will be less than 25 μm.

The following examples are only illustrative of certain embodiments and are not intended to limit the scope of the claimed invention.

### EXAMPLE 1

### PREPARATION OF PARA-BENZYLOXY PHENYL ACETIC ACID N-METHYL AMIDE OF FORMULA (IIIC)

Para-benzyloxy phenyl acetic acid methyl ester (Formula IIC) (144 g) was combined with monomethyl amine (700 ml) at 25-35° C. Stirred the contents at 25-35° C. for about 3-4 hours, filtered the solid, and washed with water (300 ml). Wet solid slurried in water (1000 ml) for 30 minutes and filtered the solid. Dried the solid under reduced pressure at about 45-50° C. for about 8-9 hours. The obtained compound was further purified by recrysatlization in ethyl acetate.

### EXAMPLE 2

### PREPARATION OF 3-BENZYLOXY-4-METHOXY-6-BROMOBENZALDEHYE (IIID)

108.5 g of 3-Benzyloxy-4-methoxybenzaldehyde (Formula IID) was dissolved in 1080 milliliters of methanol and 206 g of bromine was added. The reaction mass was stirred for about 2 hours followed by the addition of 1800 milliliters of water and then cooled to 5 to 10° C. and stirred for about 1 hour. The wet solid was slurred in 600 milliliters of water, filtered and washed with 400 milliliters of water and then with 100 milliliters of toluene. Added ethyl acetate to the wet solid and heated to 80 to 85° C., stirred for about 30 minutes and cooled to 10 to 15° C. with continuous stirring for about 30 minutes. The solid was filtered and washed with 50 milliliters toluene and dried at 40 to 45° C. for about 5 hours.

### EXAMPLE 3

### PREPARATION OF PARA-BENZYLOXY-N-METHYL PHENETHYL AMINE HYDROCHLORIDE OF FORMULA (IVC)

50 g of the compound of Formula (IIIC) (obtained by the method of Example 1) was combined with 1,4-dioxane (700 ml) and stirred for 15-20 minutes. Sodium borohydride was added to the reaction mass at about 15-20° C. Added a solution of acetic acid (80 ml) in 1,4-dioxane (260 ml) slowly at 15-20° C. Heated the reaction mass to 60-70° C. and stirred for about 5 hours. Distilled off the solvent from the reaction mass under reduced pressure. Added chloroform (750 ml) followed by water (1500 ml) to the residue at about 20-25° C. The aqueous layer was separated and extracted with chloroform (150 ml). Combined the organic layers and purged with dry HCl gas for about 12-14 hours. Distilled off the solvent under reduced pressure and cooled the residue to 0-10° C. Diethyl ether (50 ml) and methanol (50 ml) were added to the residue and stirred for about 1 hour. Filtered the separated solid and washed with petroleum ether (25 ml). Dried the solid at about 50-60° C. under reduced pressure for 6 hours.

### EXAMPLE 4

### PREPARATION OF PARA BENZYLOXY-N-METHYL PHENETHYL AMINE

The compound of Formula (IVC) (obtained from Example 3) (77 g) was added to the mixture of water (765 ml), sodium hydroxide (38.12 g), and chloroform (380 ml). Stirred the contents for about 25-30 minutes. Separated the aqueous layer and extracted with chloroform (152 ml). Combined the organic layer and used for the preparation of Example 6.

### EXAMPLE 5

### PREPARATION OF 5-BENZOYLOXY-2-BROMO-4-METHOXY BENZOIC ACID BENZOTRIAZOLE-1-YL ESTER (IVE)

The compound 3-benzoyloxy-4-methoxy-6-bromo benzoic acid (Formula IV D) (100 g), dichloromethane (1300 ml) and 1-hydroxybenzotriazole (HOBT) (49 g) were mixed at about 25-35° C. and stirred for 15 minutes. Added a solution of dicyclohexylcarbodiimide (DCC) (75 g) in dichloromethane (250 ml) over about 1-2 hours at about 25-35° C. Stirred the mixture at about 35-45° C. for about 2-4 hours. Cooled the reaction mass to 15-20° C. and filtered to remove undissolved solids followed by washing the solids with dichloromethane (200 ml). Distilled off 60-70 percent of the solvent under reduced pressure at 50-55° C. and added acetone (1000 ml). Distilled off 70-75 percent of the solvent under reduced pressure below 65° C. Cooled the reaction mass to 0-5° C. and stirred for about 2 hours. Filtered the reaction mass, washed the solid with acetone (100 ml), and dried the product at 45-50° C. under reduced pressure. (Yield 115 g)

### EXAMPLE 6

### PREPARATION OF 5-BENZYLOXY-N-[(PARA BENZYLOXY) PHENETHYL]-2-BROMO-4-METHOXY-N-METHYL BENZAMIDE (FORMULA Va)

Added a solution of compound (IVE) (100 g) (obtained in Example 5), in 1250 ml of chloroform, to a mixture of the product of Example 4 and potassium carbonate (12.5 g) at 25-30° C. over about 30-40 minutes. Stirred the mixture for about 60-90 minutes at 25-30° C. Filtered the unwanted solids from the reaction mass at 10-15° C. and washed with chloroform. Distilled off the solvent under reduced pressure at below 50° C. and cooled the residue to 25-30° C. Added a solution of sodium hydroxide (12.5 g) in methanol (375 ml), and stirred for 30-60 minutes at 25-30° C. Reaction mass cooled to 0-5° C. and stirred for 1-2 hours. Filtered the formed solids and washed with chilled methanol (100 ml) followed by chilled water. Dried the obtained compound at about 50-55° C. under reduced pressure for about 4-5 hours to get the title compound.

### EXAMPLE 7

### PREPARATION OF 5-HYDROXY-N-[(PARA-HYDROXY) PHENETHYL]-2-BROMO-4-METHOXY-N-METHYL BENZAMIDE (FORMULA V)

162.0 g of compound (Va) obtained from Example 6 was dissolved in 1620 ml of methyl isobutyl ketone, and 2106 ml of aqueous hydrochloric acid was slowly added at 30-45° C. over about 1-2 hours. Stirred the mixture at 65-70° C. for 2-4 hours. Water (810 ml) was added to the reaction mass at 25-30° C. and pH was adjusted to 2-3 with 1520 ml of caustic lye at 20-45° C. Stirred the reaction mass at 40-45° C. for 30 minutes. Aqueous layer was separated and extracted with methyl isobutyl ketone (2×812 ml). Distilled off the solvent from the combined organic layer under reduced pressure. Residue was dissolved in 500 ml of dichloromethane and cooled to 0-5° C. for about 90 minutes, and the separated solid was filtered and washed with 162 ml of dichloromethane. Dried the solid at 70-75° C. under reduced pressure for 4-5 hours.

### EXAMPLE 8

### PREPARATION OF BROMO NARWEDENONE FORMULA (VI)

2000 milliliters of chloroform was added into a round bottom flask and subjected to stirring followed by heating to reflux at a temperature of about 55° C. 36.4 g of potassium ferricyanide, 18.4 g of 5% aqueous NaHCO₃, and 10 g of the compound of formula (V) obtained from Example 7 were added to the chloroform at a temperature of about 55 to 57° C. with simultaneous stirring. The reaction mass was then cooled to a temperature of about 50° C. The reaction mass was filtered and washed with 200 milliliters of chloroform followed by the separation of the organic layer and aqueous layer. The organic layer was washed with 5% aqueous NaOH and the organic layer was separated and transferred into a round bottom flask. 250 milliliters of water was added to the organic layer and subjected to stirring followed by heating to reflux at a temperature of about 55° C. The reaction mass was then cooled to a temperature of about 30° C., and the organic and aqueous layers were separated, followed by distillation of the organic layer to a temperature of about 55° C. 30 milliliters of n-heptane was added to the reaction mass after the complete distillation of the organic layer and subjected to stirring with simultaneous cooling to a temperature of about 35° C. The solid was filtered and washed with 10 milliliters of n-heptane and subjected to suction drying.

### EXAMPLE 9

### PREPARATION OF RACEMIC BROMOGALANTHAMIDE OF FORMULA (VIA)

A compound of Formula (VI) known as bromo narwedenone (11 g) was added to tetrahydrofuran (440 ml) and cooled to −10 to 0° C. L-Selectride (72.6 ml) was slowly added over about 1-2 hour at −10 to 0° C. and the mixture was stirred for about 1-2 hours. Decomposed the reaction mass with water (110 ml) and with hydrogen peroxide solution (17.95 ml of hydrogen peroxide in 110 ml) at below 0° C. Added 10% sodium hydroxide solution (4.4 g in 44 ml water) at below 35° C. Stirred the contents for about 30-60 minutes at 25-35° C. and extracted with dichloromethane (2×110 ml). Combined organic layer was washed with sodium bicarbonate solution (11 g in 110 ml water). Distilled off the solvent under reduced pressure at below 50° C. Isolated the separated solid and washed with sodium hydroxide solution (2.2 g in 27.5 ml of water) followed by washing with water (55 ml). Dried the compound under reduced pressure at 70-75° C. for 3-4 hours.

### EXAMPLE 10

### PREPARATION OF RACEMIC GALANTAMINE (FORMULA VII)

Stirred a mixture of toluene (480 ml) and VITRIDE (63.5 g) and heated to reflux. Added slowly a solution of racemic bromo galanthamide (Formula VIA, 16.0 g, obtained by the process of Example 8) in toluene (960 ml) over about 2-3 hours at reflux and stirred for 30-60 minutes. Cooled to 0-10° C. with stirring. Added slowly 15% sodium potassium tartrate solution (49 g in 280 ml of water) at about 0-10° C. over 15-20 minutes. Filtered the reaction mass through a perlite bed and washed with toluene (75 ml). Organic layer was separated and washed with sodium chloride solution. Distilled off about 70 percent of the organic solvent under reduced pressure at 70° C. Charged methanol (160 ml) to the residue at below 60° C. and distilled off about 70 percent of the solvent, then a mixture of methyl tertiary butyl ether and dichloromethane was added and distilled off the solvents. Optionally repeated the distillation process to remove solvents in which the required compound is soluble. Finally fresh methyl tertiary butyl ether and dichloromethane was added to the residue, stirred at −5 to 0° C. for 45 minutes. Filtered the solid and washed with a mixture of chilled methyl tertiary butyl ether and dichloromethane. Dried the obtained solid under reduced pressure at 60-70° C. for 4-8 hours to get crystalline racemic galantamine.

The X-ray powder diffraction pattern of racemic galantamine was measured on a Bruker Axe, DS Advance Powder X-ray Powder Diffractometer with a Cu K alpha-1 radiation source, resulting in peaks at about 10.5, 11.0, 18.5, 18.9, 21.1, 21.3, 24.0, 27.8, and 29.4±0.5 degrees 2θ.

### EXAMPLE 11

### PREPARATION OF (−)-GALANTAMINE HYDROBROMIDE

9.25 g of racemic galantamine was dissolved in 37 ml of methanol at 30-45° C. A solution of (+)-di-para-toluoyl tartaric acid monohydrate (13.9 g) in 55.5 ml of methanol was added at 25-35° C. Stirred the contents for 20-25 hours at 25-35° C. and at reflux temperature for about 60 minutes. Cooled the reaction mass to 20-25° C. and stirred for about 60 minutes. Filtered the solid and washed with 18 ml of methanol. Solid was taken in methanol and a solution of aqueous hydrobromic acid in methanol was added slowly. Stirred the contents for about 2 hours at 25-30° C., cooled to 0-5° C. and further stirred for about 90 minutes. Filtered the solid and washed with methanol. Dried the solid at 55-60° C. for about 4-6 hours under reduced pressure to get the desired compound (4.1 g).

### EXAMPLE 12

### PURIFICATION OF (−)-GALANTAMINE HYDROBROMIDE OF FORMULA I

4.1 g of (−)-galantamine hydrobromide from Example 11 was taken into 41 ml of water and the pH was adjusted to 9-9.5 with 10% soda ash solution at 25-35° C. 3 g of sodium chloride and 41 ml of toluene were added to the reaction mass and stirred at 50-55° C. for 45 minutes. Aqueous layer was separated and washed with toluene (2×20.5 ml) at 50-55° C. Distilled off the solvent from the combined organic layers under reduced pressure at a temperature of about 65° C. Traces of solvent can optionally be removed by continuous distillation with methanol and with a mixture of methyl tertiary butyl ether and dichloromethane. Finally the residue was dissolved in a mixture of methyl tertiary butyl ether (8.2 ml) and dichloromethane (1 ml) at 50-60° C. Cooled the solution to −5 to 0° C. and stirred for 60 minutes. Filtered the solid and washed with mixture of chilled methyl tertiary butyl ether (3.3 ml) and dichloromethane (0.5 ml). Dried the solid at 50-60° C. for about 4-6 hours.

Dried solid (2.6 g) was dissolved in 9 ml of ethanol at 45-60° C. and a solution of 47% aqueous hydrobromic acid (1.5 ml) in 1.32 ml of ethanol was added slowly below 30° C. Stirred the contents for 25-30° C. for 60 minutes, filtered the solid, and washed with ethanol (2.6 ml). Dried the solid at 60-65° C. under reduced pressure.

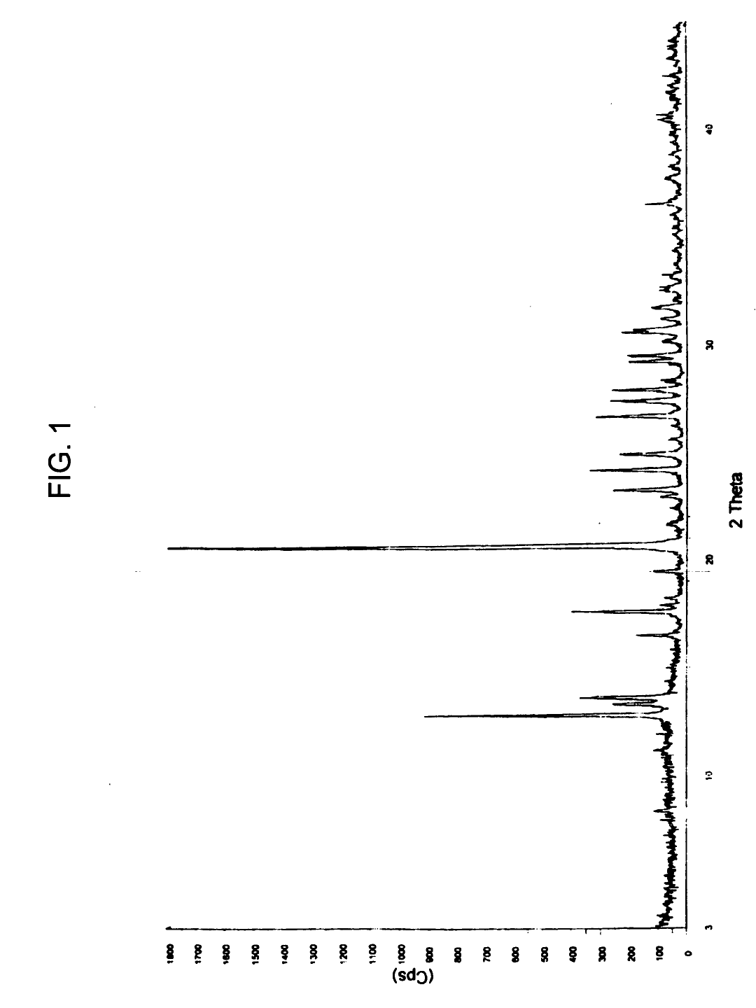

The X-ray powder diffraction pattern of the galantamine HBr was measured on a Bruker Axe, DS Advance Powder X-ray Powder Diffractometer with a Cu K alpha-1 radiation source, giving peaks at about 12.7, 13.3, 13.6, 16.5, 17.6, 19.4, 20.7, 23.2, 24.2, 24.9, 26.7, 27.4, 28.0, 29.3, 29.5, 30.7, and 31.8±0.5 degrees 2θ, as shown in FIG. 1.

### EXAMPLE 13

### STABILIZATION OF GALANTAMINE HBr (PACKAGING)

The compound was placed in a clean poly bag, which was then tied followed by placing in a black poly bag along with a silica pouch with or without an antioxidant. The black bag was filled with nitrogen, and then sealed. The black bag was placed in a triple-laminated aluminum foil bag along with a silica gel pouch, which was filled with nitrogen, and finally the bag was sealed.

Storage stability studies were done at the following different conditions: Temperature: 2-8° C.; Temperature and humidity: 25±2° C., 60±5% RH; 30±2° C., 65±5% RH; 40±2° C., 75±5% RH. There was no change in the analyses of packaged (−)-galantamine hydrobromide and no impurity content was changed under these storage conditions.

### EXAMPLE 14

### PREPARATION OF (±)(4a α,6β)-4-A,5,9,10,11,12-HEXAHYDRO-3-METHOXY-11-METHYL-11-CHLOROMETHYL-6H-BENZOFURO [3A,3,2,-EF][2] BENZAZEPINE-6-OL (FORMULA VIII)

5.0 g of compound (V) obtained from Example 7 was dissolved in 150 milliliters of dichloromethane and stirred for about 45 to 50 hours at 25 to 35° C. The solid that was obtained was filtered and washed with 2 milliliters of dichloromethane and was then suction dried and finally dried at 55 to 62° C. for about 4 to 5 hours. The ¹H NMR spectra was recorded on Mercury Plus with peaks at δ_{H} 6.82 (1H, d), 6.88 (1H, d), 2.10 (Hₐ, m), 2.30 (H_{b}, d), 5.18 (1H, br), 5.90 (1H, m), 6.20 (1H, br), 2.00-2.40 (2H, m), 4.40-4.80 (2H, m), 4.40-4.80 (Hₐ, m) 4.40-4.80 (H_{b}, m), 4.15 (1H, br), 3.75 (1H, s), 2.90 (3H, s), 5.70(2H, s).

We claim:

1. A process for preparing galantamine comprising:
a reacting bromogalantamide having the formula (VIA): 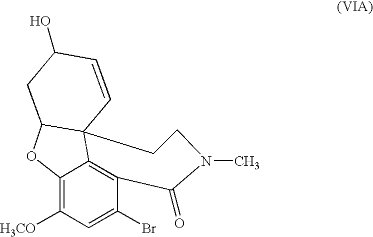
with sodium dihydro-bis-(2-methoxyethoxy) aluminate;
b adding sodium potassium tartrate solution; and
c. recovering galantamine.

2. A process for preparing (−)-galantamine hydrobromide, comprising:
a) reacting bromogalantamine having the formula (VIA): 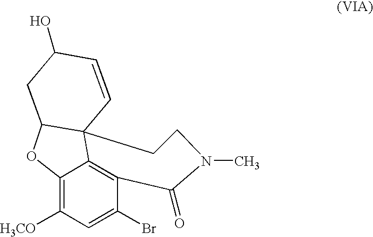
with sodium dihydro-bis-(2-methoxyethoxy) aluminate;
b) recovering galantamine;
c) reacting galantamine with an enantiomerically pure acid;
d) reacting a product from c) with hydrobromic acid; and
e) recovering (−)-galantamine hydrobromide.

3. The process of claim 2 wherein an enatiomerically pure acid is (+)-di-para-toluoyl tartaric acid monohydrate.

4. The process of claim 2 wherein recovered (−)-galantamine hydrobromide contains less than about 0.2 percent by weight of an impurity having formula (VIII):

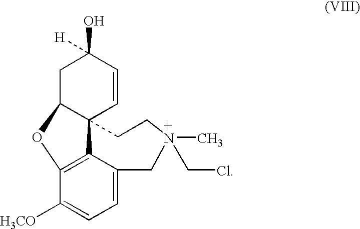

5. The process of claim 2 wherein recovered (−)-galantamine hydrobromide contains less than about 0.15 area-percent, as determined by high performance liquid chromatography, of an impurity having the formula:

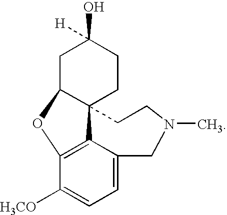

6. The process of claim 2 wherein recovered (−)-galantamine hydrobromide contains less than about 0.15 area-percent, as determined by high performance liquid chromatography, of an impurity having the formula:

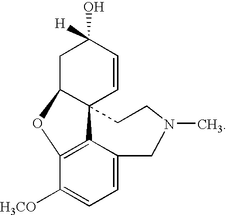

7. The process of claim 2 wherein recovered (−)-galantamine hydrobromide contains less than about 0.15 area-percent, as determined by high performance liquid chromatography, of an impurity having the formula:

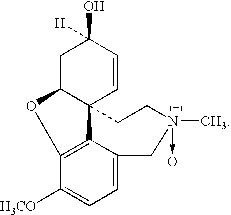

8. The process of claim 2 wherein recovered (−)-galantamine hydrobromide contains less than about 0.15 area-percent, as determined by high performance liquid chromatography, of an impurity having the formula:

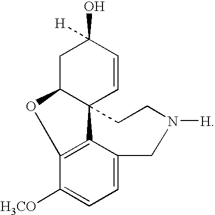

9. The process of claim 2, wherein recovered (−)-galantamine hydrobromide contains less than about 1 area-percent, as determined by high performance liquid chromatography, of impurities.

10. A process for preparing a compound having formula (VA): 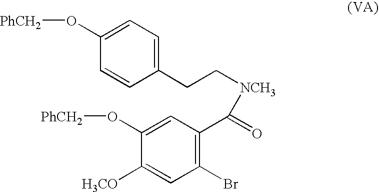
comprising:
a) reacting 3-benzoyloxy-4-methoxy-6-bromobenzoic acid with 1-hydroxybenzotriazole, in the presence of dicyclohexylcarbodiimide;
b) reacting the product from a) with p-benzyloxy-N-methylphenethylamine hydrochloride; and
c) recovering a product having formula (VA).

11. A process for preparing a compound having formula (IIIC): 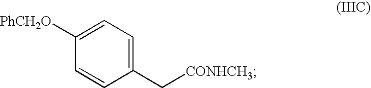
comprising reacting a compound having Formula (IIC) 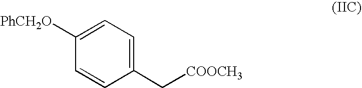
with methylamine.

12. A process for preparing a compound having formula (IVC): 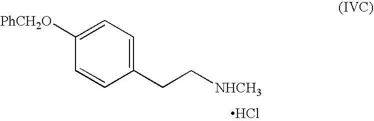
comprising:
a) reacting a compound having formula (IIIC): 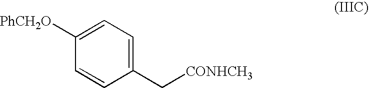
with NaBH₄; and
b) reacting a product from a) with HCl.

13. A process for preparing a compound having formula (IIID): 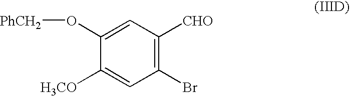
comprising: reacting a compound having formula (IID): 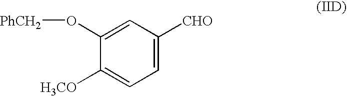
with bromine.
